(19) United States
(12) Patent Application Publication
(10) Pub. No.: US 2014/0194740 A1
(43) Pub. Date: Jul. 10, 2014

(54) Emboli detection in the brain using a transcranial doppler photoacoustic device capable of vasculature and perfusion measurement

(71) Applicants: Stuart Stein, Santa Ana, CA (US); Carl Pennypacker, El Cerrito, CA (US)

(72) Inventors: Stuart Stein, Santa Ana, CA (US); Carl Pennypacker, El Cerrito, CA (US)

(73) Assignee: Cerebrosonics, LLC, Tucson, AZ (US)

(21) Appl. No.: 14/070,264

(22) Filed: Nov. 1, 2013

Related U.S. Application Data
(60) Provisional application No. 61/833,802, filed on Jun. 11, 2013.
(60) Provisional application No. 61/749,618, filed on Jan. 7, 2013.

(30) Foreign Application Priority Data
Oct. 31, 2013 (US) PCT/US2013/066713

(51) Int. Cl. A61B 8/08; A61B 8/06

(52) U.S. Cl. CPC A61B 8/085; A61B 8/06; A61B 8/488
USPC 600/455; 600/454

(57) ABSTRACT

A device, method, and system for detecting emboli in the brain is disclosed. A transcranial Doppler photoacoustic device transmits a first energy to a region of interest at an internal site of a subject to produce an image and blood flow velocities of a region of interest by outputting an optical excitation energy to said region of interest and heating said region, causing a transient thermoelastic expansion and produce a wideband ultrasonic emission. Detectors receive the wideband ultrasonic emission and then generate an image of said region of interest from said wideband ultrasonic emission. A Doppler ultrasound signal will also be deployed to image the region of interest. Doppler presents changes in velocity to map blood flow. Additionally, a dye can be given to visualize the brain vasculature and a perfusion measurement can be made in various regions of the brain along with the transcranial Doppler and the photoacoustic screening. Systems are taught using resultory medical data for better triage within an enhanced stroke ecosystem.

### FIELD OF THE DISCLOSURE

The present inventions relate to use of medical imaging to classify and support lumen-challenged patients. In particular, the instant disclosure relates to a carotid Doppler, transcranial Doppler, phased array, photoacoustic device that provides remote wireless monitoring and remote control of the sensors or transducers of this device to and from a data/stroke center, staffed by experts, and method that will produce a more complete picture of a traumatic event in the brain, for example, a stroke, or cerebrovascular accident (CVA) or predilection to said condition. In the acute care setting of stroke and particularly in the prehospital situation for patients, in ambulances, helicopter, or airplanes, this invention will aid neurologists, radiologists and stroke teams by simultaneously obtaining rapid blood velocity measurements in neck vessels and brain vessels, determination of neck and brain large blood vessel, acute blockage or narrowing, and obtaining information on irreversibly injured brain versus potentially reversible brain at the stroke site.

The present disclosures, independently, and in combination with neurological evaluation, stroke protocols, and other imaging studies may influence the timing of delivery and appropriateness of therapeutic agents, as well as optimum location of care and services. The current disclosure in combination with stroke telemedicine, deployed in prehospital settings, may provide physiological and clinical information that can be critical for decision making in acute stroke.

Furthermore, the present device can aid the diagnosis and prevention of stroke, by remote screening of and identification of patients at risk for stroke related to intercurrent medical problems and previous stroke that will provide medical doctors and neurologists with information to implement the best treatment plan. The current invention can be added to the current chain of acute stroke care to augment and produce a unified stroke ecosystem.

### BACKGROUND OF THE DISCLOSURE

Stroke affects approximately 795,000 Americans each year, and approximately 6.4 million stroke survivors are now living in the United States. Although progress has been made in reducing stroke mortality, it is the fourth leading cause of death in the United States. Moreover, stroke is the leading cause of disability in the United States and the rest. of the world: 20% of survivors still require institutional care after 3 months and 15% to 30% experience permanent disability. Stroke is a life-changing event that also affects the patient's family members and caregivers. With an aging US population tending to heavier body weights, the situation will only become more significant with regard to stroke.

Stroke is a global problem: In Germany, the projections for the period 2006 to 2025 showed 1.5 million and 1.9 million new cases of ischemic stroke in men and women, respectively, at a present value of 51.5 and 57.1 billion EUR, respectively. In Europe, stroke occurs in 7.2 individuals per 1000 per year (In Germany, 350 people per 100,000 population have a stroke yearly) with a short term mortality rate of 12%. This rises with age and with certain races and countries, being disproportionately higher in China, Africa, and South America, where stroke mortality may be as high as 27%. The risk for initial stroke may be significantly higher in patients with hypertension, diabetes, obesity, prior heart attack, cardiac rhythm disturbances, including atrial fibrillation, hyperlipidemia, family history of early stroke, and smoking use arguing for active prevention.

Recurrent stroke is also related to the current risk factors and previous stroke. In developing countries, where acute stroke care is not available, preventive strategies to identify patients at risk, including those with previous strokes, may be important in reducing the occurrence of stroke and the attendant high mortality. For stroke, annual cost estimates for France and UK for stroke care are 2.5 billion Euros and 8.9 Billion pounds, which may be similar in Germany. Brain blood vessel imaging by magnetic resonance and computed tomographic imaging is expensive (thousands of dollars) and not always reimbursable or accessible. Non-invasive and affordable imaging is proactively needed to prevent and treat stroke, particularly in patients with medical disorders increasing risk and in patient that have already had a stroke and in patients with an acute stroke.

Many important factors have contributed to current understanding of stroke. The definition of transient ischemic attack (TIA) has been revised and now excludes the patient whose acute neuroimaging findings reveal ischemia even if clinical symptoms have resolved. This change has shifted some formerly classified TIA patients into the category of ischemic stroke. An ischemic stroke is the result of neuronal death due to lack of oxygen, a deficit that produces focal brain injury. This event is accompanied by tissue changes consistent with an infarction that can be identified with neuroimaging of the brain. Strokes are usually accompanied by symptoms, but they also may occur without producing clinical findings and be considered clinically silent. Additionally, many current transcranial imaging devices are severely limited by the aberrations caused by the skull or intervening tissues.

Both acute and chronic conditions may result in cerebral ischemia or stroke. Acute events that can lead to stroke include but are not limited to emboli, acute neck or brain blood vessel stenosis or occlusion, cardiac arrest, drowning, strangulation, asphyxiation, choking, carbon monoxide poisoning, and closed head injury. Further, and more commonly, the etiology of stroke is related to acute and chronic medical conditions including, embolus, vessel occlusion, large artery atherosclerosis, atrial fibrillation, left ventricular dysfunction, mechanical cardiac valves, diabetes, hypertension and hyperlipidemia.

Regardless of cause, prehospital care and triage, that may include telemedcine technologies, efficient and expeditious transfer of patients to appropriate hospitals and between hospitals, including various levels of stroke centers, prompt and accurate recognition of symptoms and neurological signs, strict adherence to established acute stroke protocols, and urgent medical attention are necessary for consideration of potential: 1) intravenous thrombolytic therapy; 2) intra-arterial thrombolytic therapy; 3) mechanical neck and cerebral artery clot removal/dissolution to be considered, evaluated, and provided.

Unlike 15 years ago, treatment(s) for acute stroke now exist, and research shows that intravenous thrombolytic therapy with tPA, improves outcomes by reducing post-stroke disability. Time, in particular, as well as the appropriate evaluation, and safety of potential interventions are the most important factors in the initiation of treatment, and this has prompted increased education and awareness campaigns for the public and emergency services providers about the signs and symptoms of stroke. When a stroke occurs, there is a loss per minute of 1.9 million neurons, 14 billion synapses or connections between neurons, and 7.5 miles of nerve fibers. Once an artery is blocked, then 80% of neurons will die within three hours. Time is brain and the earlier that appropriate treatment can be initiated, the better the outcome.

A primary treatment that has been approved by the FDA and that is routinely used in the United States and industrialized work is tPA, a thrombolytic agent, that can lyse the vessel clot when given intravenously, within 4.5 hours of stroke onset. The time window is expanded for tPA given intra-arterially. The use of this agent in selected patients and under strict criteria as defined by AHA protocol, can significantly improve neurological outcome. The earlier the tPA is administered in eligible patients, the better the outcome for walking at discharge and independent living and the reduction of mortality (Saver, 2013). However, only 27.4% of patients that are given tPA receive this within the first hour of arriving at the emergency room but in addition to the time of onset and transport emergently; therefore, because of the delays, outcome may be compromised and death risk increased.

Further of the patients that are eligible by strict criteria to receive intravenous tPA, only 4% receive tPa within the 4.5 hour time window from stroke onset and the potential patients that could be eligible for this therapy might be as high as 28% of strokes (Saver). Many patients with stroke do not get tPa or clot buster within the acceptable 4.5 hour time range, early in that time period, or do not get tPA at all. Certain hospitals in the United States and Europe can achieve Emergency room (door) to needle time of 20 to 38 minutes for patients to receive tPA, which is considered best practice and within the “golden hour” (Saver). Optimum stroke care has a “narrow therapeutic window” and “early intervention is critical” (Saver).

There are many logistical and other issues that lead to late delivery or no delivery of TPA to eligible stroke patients within the accepted time windows for treatment.

The steps for rapid and appropriate treatment include: 1) Detection with early recognition; 2) Dispatch with early EMS activation; 3) Delivery with transport and management; 4) Door with Emergency Department triage; 5) Data with ED evaluation and management; 6) Decision with neurological input and therapy selection; 7) Drug use with thrombolytic agents (at this time); 8) Disposition with admission or transfer.

These have been more specifically conceptualized. First, a complex set of events and steps need to occur. These include: 1) EMS Pre-Notification; 2) Stroke Toolkit with all things that need to be done; 3) Rapid Triage and Stroke Team Notification; 4) A Single telephone or pager Call Activation System; 5) Transfer Directly to CT scan to evaluate the brain for stroke or a bleed; 6) Rapid CT and warranted other Brain Imaging; 7) Rapid blood evaluation for blood clotting profiles and other essential measures; 8) Premixing of the clot buster for potential therapy; 9) Rapid TPA Access—store TPA in ED/radiology, start in; imaging suite; 10) Team approach; and 11) Prompt data feedback to physicians that evaluate the patient and must make the decision about tPA (Saver).

Second, the identification of stroke patients that are eligible for tPA follow strict nationally and internationally established criteria. These include an established National Institute of Health Stroke Scale examination performed by a trained physician in the Emergency Department that can evaluate the presence of stroke, the severity of the stroke, the potential brain vessel site of the stroke, and whether or not this is a stroke; many disorders, including drug overdose, repeated seizures, coma from many causes, head injury, migraine, and psychiatric disorder, may mimic stroke and these patients should not receive clot buster. Depending on the study, this can range from 5% to 50% of patients that present with a presumed stroke.

Absolute exclusions for clot buster and include but are not limited to a brain hemorrhage or bleed, severely elevated blood pressure, and current therapy with an anticoagulant agent, such as Coumadin. Inclusion and exclusion criteria are well established and also include blood and brain imaging results. Patient may not receive intravenous clot buster because of the results of these exclusion and inclusion criteria, improving or mild symptoms and signs, or arriving too late within the 4.5 hour window or after that window. There is a defined baseline risk of intravenous tPA for brain hemorrhage and this increases significantly with these exclusions, as well as other factors and conditions. Hemorrhage risk must be balanced against therapeutic potential for clot buster.

The primary purposes of this disclosure are to: 1) Reduce the time for administration of tPA or clot buster by providing physiological data on the brain and neck blood vessels and neurological examination prior to the patient reaching the Emergency Department; 2) Assist in the decision making process by Emergency Department physician providers by providing more information prior to and at the time of Emergency arrival; 3) Assist in the alerting about a potential stroke and what might need to be done and ordered prior to Emergency Department arrival; 4) Assist in the decision about where the patient might be taken in collaboration with Emergency Department physicians whether this be to a primary stroke center, a comprehensive stroke center, or the nearest emergency room, or in transfer from a primary stroke center emergency department to a comprehensive stroke center; 5) Assist in the early identification of patients with large neck or cerebral vessel obstructions, occlusions, or significant stenosis that may not respond to intravenous clot buster and that may require intra-arterial clot buster or mechanical removal of a clot at a comprehensive stroke center (see figures).

All patients with presumed acute stroke require a CT scan of the brain to evaluate for stroke and specifically to see if there is brain hemorrhage, which is an absolute criteria for not using clot buster. 6.2% of all patients that receive clot buster may have a secondary and very debilitating hemorrhage. This may increase with other medical conditions or drugs that an individual patient will have This is one of the most important reasons for the initial CT scan but also the very detailed evaluation by strict criteria of all patients. Absolute exclusions are factors related to the patient that will increase hemorrhage risk, include very high blood pressure, diabetes, or anticoagulation therapy. Once hemorrhage occurs, the morbidity and mortality rise significantly and negate positive effects of clot buster.

The risk of hemorrhage from clot buster increases in patients with obstruction by acute clot in major brain arteries, including the middle cerebral arteries and the basilar artery. Our invention can help to identify these patients with major vessel obstructions at greatest risk for hemorrhage with intravenous clot buster and that may require mechanical or intra-arterial therapy at a comprehensive stroke center or primary stroke center with interventional radiologists or interventional neuroradiologists.

Not all stroke patients should receive intravenous clot buster and may need consideration for intra-arterial clot buster or mechanical clot removal by an interventional radiologist or neuroradiologist. In a significant number of stroke centers, therapeutic decisions about intravenous clot buster and about intra-arterial clot buster are made based on rapid CT angiography to look for obstruction in major vessels. Our invention would work in combinatorial and synergistic fashion with these anatomical imaging techniques for the identification of major vessel obstructions that may require intra-arterial clot buster or mechanical removal.

### Types of Imaging in Stroke

The CT scan is essential in the evaluation for acute treatment of stroke, as it is used to rule out a brain bleed, and can in some cases be used to see a stroke. A stroke that appears on CT may represent a completed stroke and therefore, thrombolytic therapy is not warranted as there is little chance of recovery of the dead tissue and brain hemorrhage risk is increased in this context if thrombolytics are given. The sensitivity and specificity of the CT scan is increased for stroke evaluation by incorporation of the Aspect score, which is only incorporated at some centers. Beyond the CT scan, the imaging used is variable, based on time considerations, equipment availability, and philosophy.

The neurological examination with the NIH Stroke Scale Score can help to define the size of the stroke and as the size of stroke increases, the risk of hemorrhage with thrombolysis may increase. Thrombolysis is absolutely contraindicated based on large size strokes, for which there are specific criteria. A rapid MRI scan of the brain, particularly a diffusion weighted image, is very sensitive and specific for early stroke. This exam is used at some centers to evaluate stroke size and as a basis for therapy decision. However, this exam may not be available rapidly at many centers.

If allowable based on kidney function, the CT angiography of head and neck can show the major vessels and obstructions and is very useful in defining therapy. If CT angiography cannot be performed, then a MR angiogram of head and neck vessels can be performed. However, this may be more time consuming due to equipment availability and the time required to perform the test. Delays in delivery of thrombolytics to eligible patients with potential nerve cell loss must be balanced with the need for additional imaging definition, patient safety, and the need for more invasive therapy, such as intra-arterial thrombolytics or mechanical clot removal or dissolution. When CT angiography or MR angiography cannot be performed, for any reason, ultrasound techniques with definition of the neck arteries, carotid arteries and vertebral arteries, can be done with carotid Doppler for evaluation of acute and chronic stenosis and occlusion.

Transcranial Doppler can be performed to obtain physiological information about brain blood vessels, including major and large brain blood vessels, with rapid data on stenosis and occlusion and collateral blood vessel flow. Similarly, phased array methods can be used to obtain velocity and other brain blood vessel information. These studies are usually not available emergently in the United States, but may be primary parts of the acute stroke evaluation in Europe, where CT angiography, MR scans, and MR angiography may not be available. At any hospital or stroke center, the exams performed are variable and may be idiosyncratic. Both CT and MR angiography are very useful is showing collateral blood vessel flow that may determine how robust the patients vessels might be for protecting against severe damage. However, rapid data on collateral flow can be derived from TCD.

In combination with the CT angiography or MR angiography, a rapid and simple perfusion scan can be performed. The choice between CT perfusion or MR perfusion is variable across stroke centers and may not be available. Although little time is added by these studies, processing of the images may lead to delays. The value of these perfusion techniques is that they may reveal areas of dead brain tissue versus injured tissue that may be retrievable if vascular flow is restored with thrombolysis or mechanical vessel clot removal or dissolution. For MR of brain, MR diffusion weight imaging is combined with MR perfusion to evaluate dead brain tissue versus injured and potentially reversible brain tissue.

In the ideal situation, in addition to CT scan, diffusion weighted image MR scan of brain, CT or MR angiography, and CT perfusion or MR perfusion might be useful, but the essential nature of all exams is variably accepted from a philosophical and practical standpoint. All require more time. Any measure that will provide rapid additional information that is complimentary or can replace some of the above imaging testing and that can be done with less time is useful. Ultrasound and photoacoustic technologies, as incorporated in this intervention, provide alternatives to compliment data for patient decision making that can complement centers where only CT scans are improved, where time is at a premium as the 4.5 hour thrombolysis window is approach. Our invention provides rapid alternatives of independent and complimentary value to other imaging techniques for evaluating acute brain and neck blood vessel occlusion or narrowing and for potentially revealing areas of dead brain tissue versus potentially viable injured tissue contiguously.

Despite conflicting studies in the literature, the initiation of intra-arterial clot buster through a catheter by specialized professionals and/or mechanical removal of major vessel clots, particularly with devices called stent retrievers, may improve morbidity and mortality in selected patients; this is seen, particularly, in those patients with acute obstruction, stenosis, or occlusion of major arteries, such as the carotid arteries, middle cerebral arteries, basilar artery, or vertebral arteries. These devices or intra-arterial clot buster may lead to reopening or recanalization of acutely blocked blood vessels. The devices include catheter based clot retrieval devices, clot suction devices or mechanical clot disruption devices. Many of these devices require advancing a catheter with device through the arteries in the groin and then up to the neck and brain blood vessels. In addition but not limited to the stroke severity, the success of these therapies depend on the vessel and its size and the location of the clot within the vessel, the size of the clot within the vessel, whether the clot completely or partially obstructs the vessel, the time of the procedure after stroke onset.

Although intravenous clot buster may reduce mortality and morbidity, it is not always efficacious in removing the clot blocking a vessel. Intravenous clot buster may give 40% partial vessel opening or recanalization and 5% complete opening. Intra-arterial clot buster may lead to 65% partial opening and 20% complete opening. These percentages of partial and complete opening are improved with intra-arterial clot removal or dissolution devices, but particularly with a series of devices called stent retrievers, i.e. Trevo and solitaire. The solitaire device may achieve 93% partial recanalization and 66% with complete vessel opening (Saver, Feinberg lecture). Improved later disability may be seen in selected patients (Saver). Further, intra-arterial therapy or mechanical re-opening therapies may extend the time window for treatment from 6 to 12 hours in some stroke cases (personal communication, Mao, 2013). This has been applied in some centers with positive results. However, the use of these mechanical reopening devices may have hemorrhage and additional stroke risk that must be balanced with potential benefits for the patient.

The value of our device is in the early identification of potential patients with major neck or brain artery acute occlusions or blockages in the ambulance, helicopter, or fixed window airplane, that can then be considered for rapid transport to a comprehensive stroke center or primary stroke center where both have quality interventional radiology or interventional neuroradiology for clot dissolution or removal.

Further integration of the stroke ecosystem of care for improvement in identification, efficiency, appropriate site of transfer, earlier alerting to appropriate specialists, earlier and more appropriate use of intravenous clot buster and intra-arterial clot buster or mechanical reopening strategies, safety due to additional information before clot buster given at places where only CT is done with no vascular imaging. Acute stroke care has improved related to an increasing number of stroke ready hospitals, certified primary stroke centers, and certified comprehensive stroke centers.

Prehospital Identification and Treatment of Stroke. The earlier identification and evaluation and initiation of therapy or place to transport has the potential for improving morbidity and mortality and in the pre-identification of patient for tPA. The time from pickup in an ambulance, helicopter, or fixed wing airplane may range from 10-15 minutes in certain urban centers with integrated ambulance services to up to 1 hour within New York City. Rural sites may allow rapid transit to local Emergency departments, but these facilities may have a CT scanner, but do not have other specialists or diagnostic services.

Nevertheless, these facilities can transport emergently under certain circumstances with consultation with a stroke center with tPA given as a drip and ship protocol. The time in transport from a rural or distant facility to a stroke center may be greater than 30 minutes and usually longer. The time in the ambulance, helicopter, or fixed winged airplane even when short affords a window for rapid evaluation with neurological examination, i.e. LA motor scale or NIH stroke scale by stroke telemedicine, or physiological measures, i.e. transcranial Doppler or carotid Doppler.

The Regensburg ambulance trial has used neurological or emergency physicians riding in the ambulance to identify stroke and perform transcranial Doppler in the ambulance. Transcranial Doppler is very useful in evaluating vessels rapidly for occlusion and stenosis. (Hoelcher).

The Berlin Stroke ambulance of Charite Hospital travels with a CT scan and has blood laboratory and data is obtained in the ambulance and decisions within the ambulance and also remotely during transport can lead to the delivery of TPA within the ambulance in elgible patients without hemorrhage. However, the utility of having an expensive CT scanner in an ambulance, a neurologist or other physician in the ambulance, and the potential lack of vascular imaging may limit this approach in the United States and other venues. This has led to earlier treatment with intravenous TPA in the ambulance versus waiting for emergency department evaluation and treatment (Walter, 2012).

Stroke telemedicine involves the use of video and audio systems, often with a moveable robot at the hospital where the acute stroke patient is being evaluated. Experts or neurologists can be rapidly and emergently connected to the hospital where the acute stroke patient is located. Stroke telemedicine has proven useful in neurological evaluation and NIH stroke scale performance and in concert with imaging transmitted to experts at the remote Stroke teledicine center, decisions can be made about clot buster. Experts or neurologists that staff the stroke telemedicine center or service often take the calls at home and evaluate the patient on desktop or laptop computers. A robot cannot be used in the ambulance but a video device with connection to a stroke telemedicine center is useful. A portable device has recently been used in an ambulance for acute stroke patient evaluation in Belgium. Stroke telemedicine capacities can supplant primary or comprehensive stroke personnel/physicians and provide rapid evaluation in remote locations or in time critical situations when local experts are not available.

Telestroke can also provide a means for triage decisions within an ambulance, helicopter, or fixed wing airplane if the device if the video and audio device is portable and appropriate criteria can be established (Higishida). However, stroke telemedicine in ambulances and the like is not routine and can be technically difficult and expensive. Equipment is not uniformly available, has not been appropriately designed, and logistic issues have not been addressed.

The utility of combining the instant disclosure with a video and audio device and other features that can provide stroke telemedicine capacities, all within a remote stroke data center, with wireless connection, bidirectional interchange with emergency personnel in an ambulance or the like is evident. This combination of physiological and neurological evaluation would be and can be time saving, lead to appropriate triage and transport to specific stroke center levels, and provide additional information for what needs to be set up a the receiving facility. There are additional advantages to our device/invention that have been documented above (Please put in section)

Stroke Telemedicine. When acute stroke care is not available either because of regional limitations, lack of resources, or time of day, telestroke options can be evoked with linkage to stroke telemedicine centers staffed by expert neurologists that can evaluate the patient, perform a neurological examination, including a formal NIH stroke scale evaluation, and also prescribe clot buster or other therapies in concert with a healthcare facility where imaging including CT scan and/other imaging modalities for stroke might be available. Organized networks based on a hub and spoke model with evaluation at the hub have been successfully utilized for acute stroke in the United States, which is expanding, and are routine for acute stroke care delivery in some regions and cities around the United States. A stroke robot can act as a mobile two way communication vehicle including sound and video input for acute patient evaluation rapidly in an emergency department or other hospital acute care setting, i.e. ICU. This is also linked with a stroke system of care, where the experts at the remote center have the ability to view CT scans and other radiological examinations directly. In Belgium and in Germany, trials are underway and have shown efficacy of using a video and audio system for stroke telemedicine within an ambulance.

Robots are too large for an ambulance. Stroke telemedicine will allow accurate evaluation of stroke versus mimicers and diffentiation from anterior versus posterior circulation strokes, as well as size of stroke and potential vessel size and identity. In our invention, one emboldiment of the stroke ecosystem would be the use of our distant data center/stroke center to evaluate patients in the ambulance, helicopter, or fixed wing with telestroke neurological examination and NIH stroke scale performance in combination with physiological data from TCD, phased array, carotid Doppler, and photoacoustic.

According to our disclosures, embodiments of the stroke ecosystem would be the use of our distant data center/stroke center to evaluate patients in the ambulance, helicopter, or fixed wing with bidirectional interchange with emergency personnel in an ambulance or the like and the patient, wireless data flow for imaging, video, and audio information, telestroke neurological examination and NIH stroke scale performance in combination with remote monitoring and remotely controlled delivery of physiological data from TCD, phased array, carotid Doppler, and photoacoustic imaging. This combination of physiological and neurological evaluation would be and can be time saving, lead to appropriate triage and transport to specific stroke center levels, provide additional information for what needs to be set up a the receiving facility. This represents important pieces of a unified stroke ecosystem that may improve care quality, patient safety, and reduce costs

The arrival of a stroke patient in the emergency room (ER) must be viewed as a true emergency, and the urgent care of such patients should receive absolute priority. On arrival to the ER, identification of the patient with a potential stroke should prompt the collection of several important data points: 1. time the patient was last known to be neurologically normal; 2. detailed neurological exam and the use of National Institutes of Health Stroke Scale (NIHSS); 3. serum glucose level; 4. medical history; and, 5. current medications.

At the time of stroke, mini-strokes, suspected strokes, or TIAs, magnetic resonance (MRI) and computed tomography (CT) of the brain (intracranial) and neck are the most employed techniques/devices used to look for blood vessel abnormalities, including stenosis, as a basis for stroke. These techniques are expensive, inaccessible to rural health care centers, and are time consuming, and the sequence of testing has been discussed above.

Hence, existing modalities to Image the brain and its blood flow require a substantial financial outlay by a healthcare provider and none, including CT and MR testing, can perform real time analysis of blood flow velocity and flow direction, detect and characterize emboli and measure vessel-wall thickness. In addition to CT and MR testing, positron tomography (PET) and SPECT scanning may provide useful information, but are not accessible and not generally available. The use of modalities in acute stroke care has been discussed above.

Stroke or transient ischemic attacks (TIA) involve brain tissue damage that is permanent (stroke) or transient (TIA) from the obliteration of blood flow with reduced oxygen delivery through specific extracranial vessels, i.e. carotid arteries, cervical arteries, vertebral arteries, or intracranial vessels, i.e. middle cerebral arteries, posterior cerebral arteries due to atherosclerotic vessel change, emboli, or a combination of both. Emboli may be gaseous or particulate. The latter may involve calcium, fat, and blood elements including platelet, red blood cells, or organized clot, i.e. thrombin with platelets or thrombin alone.

The size of these embolic components is approximately 50 microns for particulate or solid emboli and 1-10 microns for gaseous emboli. Particulate emboli may have a more important role in stroke or TIA causation, as compared to gas emboli; this underlies a need for detection and differentiation of particulate versus gas emboli.

Cerebral emboli may be associated with cardiac, aorta, neck and intracranial vessel disease, as well as coagulation disorders and neck and during diagnostic and surgical procedures on the heart and the carotid arteries. Cerebral embolism can be a dynamic process episodic, persistent, symptomatic, asymptomatic, and may, but, not in all cases, predispose to stroke or TIA, influenced to some degree by composition and size; the latter embolic stroke, which is influenced by the vessel and its diameter to which the embolus goes. Transcranial Doppler can detect emboli, but strict criteria must be applied, as artifact and false positives may be detected instead of real emboli.

### Transcranial Doppler

Transcranial Doppler (TCD) is a test that measures the velocity of blood flow through the brain's blood vessels. Used to help in the diagnosis of emboli, stenosis, vasospasm from a subarachnoid hemorrhage (bleeding from a ruptured aneurysm), and other problems, this relatively quick and inexpensive test is growing in popularity in the United States. The equipment used for these tests is becoming increasingly portable, making it possible for a clinician to travel to a hospital, doctor's office or nursing home for both inpatient and outpatient studies. It is often used in conjunction with other tests such as MRI, MRA, carotid duplex ultrasound and CT scans.

Transcranial Doppler provides a basis, alone and taken together (with other tests) for determining occlusion or vessel stenosis for major and multiple brain blood vessels, collateral flow in the brain, determination of re-canalization or re-occlusion during and after clot buster or mechanical intervention, putative emboli and potential treatment planning and initiation. An embodiment of the present invention allows for multiple brain blood vessels can be evaluated simultaneously and the skull can be sampled at multiple points. Since this technique can be difficult even in expert hands, its application within the ambulance will involve transducer positioning over the necessary neck and brain blood arteries by remote monitoring and remote control with positioning for quality and maximal signals.

TCD can be used to look at velocity measures and resistance in multiple cerebral vessels in the front (anterior) and back (posterior) circulation of the brain. Occlusion and stenosis can be deduced from changes in velocity, up or down versus the opposite side or based on age related norms, and the waveform anatomy of a vessel (normal versus varying degrees of flatness or change in normal anatomy). TCD cannot directly visualize vessel anatomy but different depths of a vessel and their velocity and resistance and waveform characteristics can be looked at. Using TCD, emboli by strict criteria including auditory, visual, intensity above background, unidirectionality, and randomness and adherence to accepted guidelines.

Transcranial Doppler provides a basis, individually and taken together (with other tests) for decision making that may impact on treatment planning and initiation, applied to acute and preventive stroke care. An embodiment of the present invention allows for multiple brain blood vessels can be evaluated simultaneously or serially, using ultrasound transducers, mounted in a helmet. The position of these transducers to promote ideal blood vessel detection is remotely controlled from a distant data and stroke center by experts including neurologists and radiologists in the cloud. The remote control uses robotic software and hardware technology and micromotors to control probe position under control from the remote data and stroke center.

The raw ultrasound data from the transcranial Doppler insonation is also transmitted wirelessly to the remote data and stroke center for processing, analysis, and storage. Multiple cerebral vessels can be sampled by the position of the transducer. In our embodiment, both middle cerebral arteries and anterior cerebral arteries and posterior cerebral arteries as well as collateral arteries at the temporal bone windows, both ophthalmic arteries from on top of the eyelids, the basilar artery and both vertebral arteries from the back of the skull, and many other arteries in the brain anterior and posterior circulation can be insonated rapidly, including the distal internal carotid arteries. The placement of the transducers can be sampled at many sites without concern for skull bone interference, but some sites may have skull bone interference which can be addressed by the positioning and placement of probes.

Carotid Doppler represents an ultrasound technique that has been routinely used to non-invasively evaluate the bilateral common carotid arteries, the extent of the bilateral internal carotid arteries within the neck, and the bilateral vertebral arteries in the neck. Chronic and acute stenosis and occlusion, velocity measures, atherosclerosis, wall thickness (reflecting on intimal media thickness) as well as plaque characterization can be determined from carotid Doppler evaluation. As part of the current device/invention, the biplanar carotid has b mode, pulsed wave, color flow monitoring, power Doppler, m mode and automatic measurement. Echogenicity is measured as well as velocity. The full extent of the neck internal carotid artery and common carotid artery can be imaged with linear and concave transducers. Acute stenosis and occlusion utilize these functions of the device that reveal well established velocity parameters and ratios for stenosis and occlusion.

Blood flow velocity is recorded by emitting a high-pitched sound wave from the ultrasound probe, which then bounces off of various materials to be measured by the same probe. A specific frequency is used (usually close to 2 MHz), and the speed of the blood in relation to the probe causes a frequency shift, wherein the frequency is increased or decreased. This frequency change directly correlates with the speed of the blood, which is then recorded electronically for later analysis. Normally a range of depths and angles must be measured to ascertain the correct velocities, as recording from an angle to the blood vessel yields an artificially low velocity.

Because the bones of the skull block the transmission of ultrasound, regions with thinner walls insonation windows must be used for analyzing. For this reason, recording is performed in the temporal region above the cheekbone/zygomatic arch, through the eyes, below the jaw, and from the back of the head. Patient age, gender, race and other factors affect bone thickness, making some examinations more difficult or even impossible. Most can still be performed to obtain acceptable responses, sometimes requiring using alternate sites from which to view the vessels.

### Carotid Doppler

### Photoacoustic Spectroscopy

Photoacoustic spectroscopy is the measurement of the effect of absorbed electromagnetic energy (particularly of light) on matter by means of acoustic detection. The discovery of the photoacoustic effect dates to 1880 when Alexander Graham Bell showed that thin discs emitted sound when exposed to a beam of sunlight that was rapidly interrupted with a rotating slotted disk. The absorbed energy from the light is transformed into kinetic energy of the sample by energy exchange processes. This results in local heating and thus a pressure wave or sound. Later Bell showed that materials exposed to the non-visible portions of the solar spectrum (i.e., the infrared and the ultraviolet) can also produce sounds. 1) Photoacoustic imaging is a well-known art that uses a light beam, typically a laser, to deposit energy molecules and absorption bands the converted energy from the laser to be reemitted as thermal energy to create an expansion of the materials around the molecule, and create a pressure wave. Such pressure waves are in vivo, and cause mechanical disturbances of the media, and manifest themselves partially as broadband ultrasound.

Ultrasound typically permeates and scatters much less than optical light, so photoacoustic imaging then gives one the capability to resolve structures with much higher resolution than simple measuring the back-scattering of light, particularly in soft tissues. With respect to photoacoustic imaging in the skull, one is limited by the scattering of ultrasound in the skull. For skull windows, this might result in smearing of the ultrasound resolution by a typical thickness parameter of the skull window, which may be a few millimeters. Hence, one probably will never be able to attain the same resolution in the brain with ultrasound as one does compared to targets under comparable distances of flesh, but if one is searching for major arteries and veins and large structures, such as a major hemorrhage, such resolution better than a few millimeters is not useful.

Photoacoustic imaging is based on the photoacoustic effect. In photoacoustic imaging, non-ionizing laser pulses are delivered into biological tissues (when radio frequency pulses are used, the technology is referred to as thermoaccoustic imaging). Some of the delivered energy will be absorbed and converted into heat, leading to transient thermoelastic expansion and thus wideband (e.g. MHz) ultrasonic emission.

The generated ultrasonic waves are then detected by ultrasonic transducers to form images. It is known that optical absorption is closely associated with physiological properties, such as hemoglobin concentration and oxygen saturation.

Hemoglobin (Hb or Hgb) is the iron-containing oxygen-transport metalloprotein in the red blood cells of most vertebrates. Hemoglobin in the blood carries oxygen from the respiratory organs (lungs or gills) to the rest of the body (i.e. the tissues) where it releases the oxygen to burn nutrients to provide energy to power the functions of the organism, and collects the resultant carbon dioxide to bring it back to the respiratory organs to be dispensed from the organism.

In general, hemoglobin can be saturated with oxygen molecules (oxyhemoglobin), or desaturated with oxygen molecules (deoxyhemoglobin). Oxyhemoglobin is formed during physiological respiration when oxygen binds to the heme component of the protein hemoglobin in red blood cells. This process occurs in the pulmonary capillaries adjacent to the alveoli of the lungs. The oxygen then travels through the blood stream to be dropped off at cells where it is utilized as a terminal electron acceptor in the production of A TP by the process of oxidative phosphorylation. It does not, however, help to counteract a decrease in blood pH. Ventilation, or breathing, may reverse this condition by removal of carbon dioxide, thus causing a shift up in pH.

### Vasculature and Perfusion Measurement

Perfusion is the process of delivery of blood to a capillary bed in the biological tissue. Vasculature and perfusion measurements in the Brain perfusion (more correctly transit times) can be estimated with contrast-enhanced computed tomography or MR perfusion. To get a better representation of the blood flow in the brain, a dye is injected into the patient to enhance visualization of the suspect area.

Tissue plasminogen activator (tPA) is used in diseases that feature blood clots, such as stroke, pulmonary embolism, myocardial infarction, and stroke, in a medical treatment called thrombolysis. To be most effective in ischemic stroke, tPA must be administered as early as possible after the onset of symptoms. Protocol guidelines require its use intravenously within the first three hours of the event, after which its detriments may outweigh its benefits. tP A can either be administered systemically or administered through an arterial catheter directly to the site of occlusion in the case of peripheral arterial thrombi and thrombi in the proximal deep veins of the leg.

Therefore, what is needed is a medical device with TCD, Photoacoustic, phased array and Fluorescent Dye for determination of Cerebral Blood Flow, Oxygenation and is capable of providing information on brain perfusion, which may be impaired in stroke. CT Perfusion and MR perfusion with diffusion weight MRI are helpful in providing potential definition of completed stroke or infarct versus injured but potentially retrievable tissue, the penumbra. Our disclosure is capable of defining Diffusion/Perfusion Match/Mismatch which would aid in the determination for tPA usage where the presence and amount of retrievable tissue versus completed stroke is important. It would also be beneficial that a device and method could be deployed in a rural, urban, clinic, third world or be an inpatient device for Brain Blood Flow, Vessel Definition, Emboli Detection and Brain Metabolism.

Phased Array: Phased Array Ultrasound is a well-known art that enables the use of multiple transducers to be pulsed and readout independently. Having an array of such devices enables beam steering, beam forming, and higher resolution imaging upon return of the reflected/scattered ultrasound, due to the larger effective receiving aperture. The beam can be electronically steered, and then read back for that part of space interrogated by the smaller beam size enabled by the phased array beam-forming algorithms. Such devices are used in Medical Imaging and in many industrial applications.

Typically, because of the much higher resolution afforded by MRI and CT scanning devices, phase array ultrasound has not been used in the brain. However, when larger structures are imaged, such as major vasculature, and superb resolution is not desired, phased array ultrasound is adequate. In particular, phased array ultrasound can fit into a small box, of size 10″×10″×3″, and be part of an ambulances or Emergency Department or other medical settings equipment, as compared to the room-size MRI's and CT scanning systems. The beam steering function may also allow visualization of additional brain blood vessels than transcranial Doppler. Phased array can also be utilized, as with transcranial Doppler to look at the anterior and posterior circulation vessels of the brain.

### Objects and Summary of the Disclosure

The present inventors have discovered that mapping blood flow in the brain, including by velocity, enables rapid determination of stroke and related brain insults and injuries. Devices, system and apparatus using such blood flow mapping (captured as resultory medical data) enable rapid classification, transport and selection of treatment options for patients.

According to embodiments, there is disclosed a device for detecting emboli in the brain comprising, in combination: an array of ultrasound transducers; actuators coupled to the array of ultrasound transducers; said actuators enabled to alter, skew, move, rotate, or change the position of the transducers; said actuators enabled to be controlled remotely; and the array of ultrasound transducers having the ability to learn and send Doppler shifted signals, regarding blood flow from brain and neck vasculatures, to a remote site.

According to embodiments, there is disclosed a method for detecting emboli in the brain and sending the relevant data to a remote site comprising: configuring an ultrasound array to transmit a beam pattern sufficient to isonate a region of interest at an internal site of a subject; finding, creating, and displaying maps or images of said region of interest; identifying acute occlusion or stenosis in major brain and neck arteries; wirelessly transmitting data identified to a remote site; and wirelessly receiving information about data identified from the remote site at the site of the subject where the device is being used.

According to embodiments, there is disclosed a method of configuring an ultrasound array to transmit a beam pattern sufficient to insonate a region of interest at an internal site of a subject, said method comprising the steps of: a) providing an array of ultrasound transducer elements; b) outputting a beam pattern from said array of ultrasound transducer elements to insonate a region of interest at an internal site in a body sufficiently large that the beam pattern comprises a multi-beam pattern, insonating multiple receiver elements over a substantially simultaneous period by directing energy produced by said array of ultrasound transducer elements into said region of interest in said body, and adjusting an amplitude of energy output by said array of transducers to cause the beam pattern output to have a generally flat upper pattern and nulling in a grating lobe region; and, c) introducing a phase shift of the beam pattern output from said array of ultrasound transducer elements, wherein a degree of phase shift increases as a distance increases from a central output area of said array of ultrasound transducer elements.

According to embodiments, there is disclosed a method of configuring an ultrasound array to transmit a beam pattern sufficient to insonate a region of interest at an internal site of a subject, said method comprising the steps of: a) providing an array of ultrasound transducer elements; b) outputting a beam pattern from said array of ultrasound transducer elements to insonate a region of interest at an internal site in a body sufficiently large that the beam pattern comprises a multi-beam pattern, insonating multiple receiver elements over a substantially simultaneous period by directing energy produced by said array of ultrasound transducer elements into said region of interest in said body, and adjusting an amplitude of energy output by said array of transducers to cause the beam pattern output to have a generally flat upper pattern and nulls in a grating lobe region; and, c) introducing a phase shift of the beam pattern output from said array of ultrasound transducer elements, wherein a degree of phase shift increases as a distance increases from a central output area of said array of ultrasound transducer elements.

According to embodiments, there is disclosed a method for operating an array of ultrasound transducer elements, wherein: the element spacing in the array is greater than, equal to or less than a half wavelength of the ultrasound energy produced by the elements, and, wherein the array is used differently in transmit and receive modes, comprising: forming a transmit beam from a position external to a region of interest encompassing a plurality of receive beams and initially acquiring a signal by insonating a target region comprising multiple receive beam positions over a substantially simultaneous period; receiving data from the multiple receive beam positions of the array; combining the received data in a processor; locking onto the receive beam and the point(s) producing a peak signal; and correcting for motions in the target region by periodically forming multiple receive beams and re-acquiring the peak signal.

According to embodiments, there is disclosed a method to detect prostate cancer, comprising: injecting ICN-pSMA molecule to the prostate area which combines with the surface of Prostate Cancer cells, and liberates ICN in the process; insonating the prostate and the adjacent vicinity with energy from a phased array Doppler device; insonating the prostate and the adjacent vicinity with energy from a photoacoustic device; and detecting the spectrum of a plurality of ICN molecules in the region of the Cancer.

### BRIEF DESCRIPTION OF THE DRAWINGS

The above and other exemplary features and advantages of certain exemplary embodiments of the present invention will become more apparent from the following detailed description of certain exemplary embodiments thereof when taken in conjunction with the accompanying drawings, in which:

FIG. 1 illustrates a flow diagram of a system comprising the Helmet Device in accordance with an aspect of the invention.

FIG. 2 illustrates a flow diagram overview of the Internet-Control Actuators in accordance with an aspect of the invention.

FIG. 3 is one-dimensional cross-sectional view illustrating details of the Actuators/Positioning System for the Transducers depicted in FIG. 2.

FIG. 4a illustrates a graphical view of the TCD/Actuator Module Placements on a Patients Head and their communication with a Remote Control System.

FIG. 4b illustrates a 3D view of the Trans-Cranial Doppler Device.

FIG. 5 illustrates an exemplary hierarchy chart depicting the development of a stable skull via the control box in accordance with an aspect of this invention.

FIG. 6 is an exemplary hierarchy chart illustrating the development procedure of a high-resolution 3-D model of the brain/neck arterial vasculature in accordance with an aspect of this invention.

FIG. 7 is an exemplary decision tree illustrating the process involved when incorporating the Trans-Cranial Doppler Device in an acute stroke situation.

### DETAILED DESCRIPTION

Briefly stated, a device, method, and system for detecing emboli in the brain is disclosed. The transcranial Doppler photoacoustic device transmits a first energy to a region of interest at an internal site of a subject to produce an image and blood flow velocities of a region of interest by outputting an optical excitation energy to said region of interest and heating said region, causing a transient thermoelastic expansion and produce a wideband ultrasonic emission. Detectors will receive the wideband ultrasonic emission and then generate an image of said region of interest from said wideband ultrasonic emission. A Doppler ultrasound signal will also be deployed to image the region of interest. Another embodiment Doppler would present blood flow. Additionally, a dye can be given to visualize the brain vasculature and a perfusion measurement can be made in various regions of the brain along with the transcranial Doppler and the photoacoustic screening.

Methods and apparatus of the present invention generally involve using ultrasound transducers coupled to a device, to identify, observe, and measure vasculatures in the brain. In each minute of a stroke, over a million neurons die, and over a billion synapses die. Said device thus comprises the capability to be controlled and read from a remote Data Center, thus enabling the rapid diagnosis of the type and severity of stroke a patient may be undergoing in an emergent situation. The diagnosis can then be sent to those aiding the patient, to correct the level of care for continual treatment and evaluation and care of the patient. It is a further object of this invention that remote operators can control positions of ultrasound transducers, which are mounted on motors with encoding system. A further object of this invention is that novel signal processing will allow identifying the precise location of ultrasound signal in the brain vasculature with respect to a stable coordinate system; such signals are added together from the same spot of the brain, suppressing the “noise” introduced by operator or other disturbing motions, and enable much smaller vasculature to be observed and measured. A further object of this invention is that remote neurotechnicians and neurologists can visualize in real time or data from a rapid archive the results of the TCD scan of major vasculature, and, for example, recommend transport to a comprehensive stroke center. A further object of this invention is that different types of strokes—hemorrhagic versus ischemic, for example, can be visualized and understood by Data Center staff, and appropriate measures taken. Furthermore, many other brain disorders caused by incomplete vascularization of the brain, such as Traumatic Brain Injury or Vascular Dementia can be observed and diagnosed by this system in consort with Data Center staff.

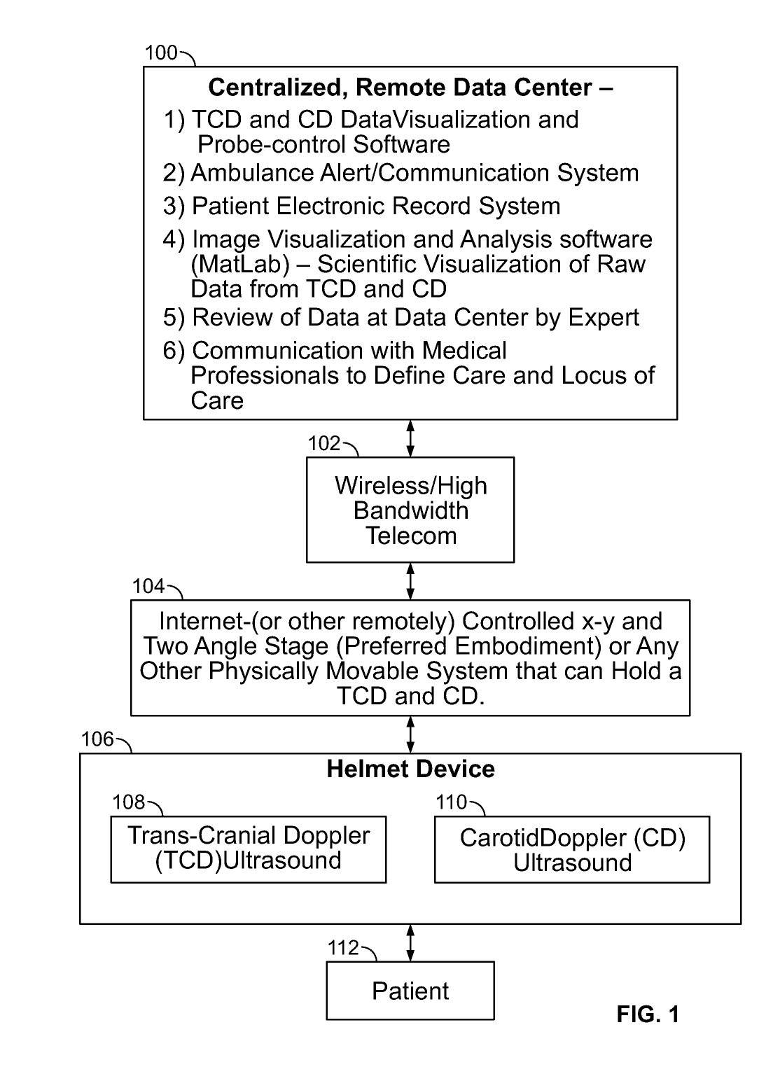

With reference now to FIG. 1, a system comprising Helmet Device is depicted in an overview. Ultrasound on areas of a patient's skull and carotid artery are done using Helmet Device 106, and information gathered from ultrasound 108, 110 are transmitted wirelessly over the Internet 104, to a Remote Data Center 100. The Helmet Device 106 is capable of operating in an ambulance, helicopter, airplane, emergency room, or the like. The advantage of the Remote Data Center 100 is that skilled technicians and doctors who well understand the hardware and software described here can be on call 24 hours a day, and respond to any stroke emergency, anywhere in the world. High-speed wireless capability exists for most modern ambulances and Emergency Rooms, with data rates of many megahertz per second available. With the ability to visualize and analyze images of patient 112 that are captured and sent wirelessly from the helmet device, physicians at the Remote Data Center 100, are then able to alert and direct the operators that are currently in person at the patients 112 side and applying the helmet device, to the most ideal and appropriate location the patient 112 should be transported to.

In an exemplary embodiment, the helmet device could be many other configurations so long as the device has the ability to comprise actuated ultrasound transducers.

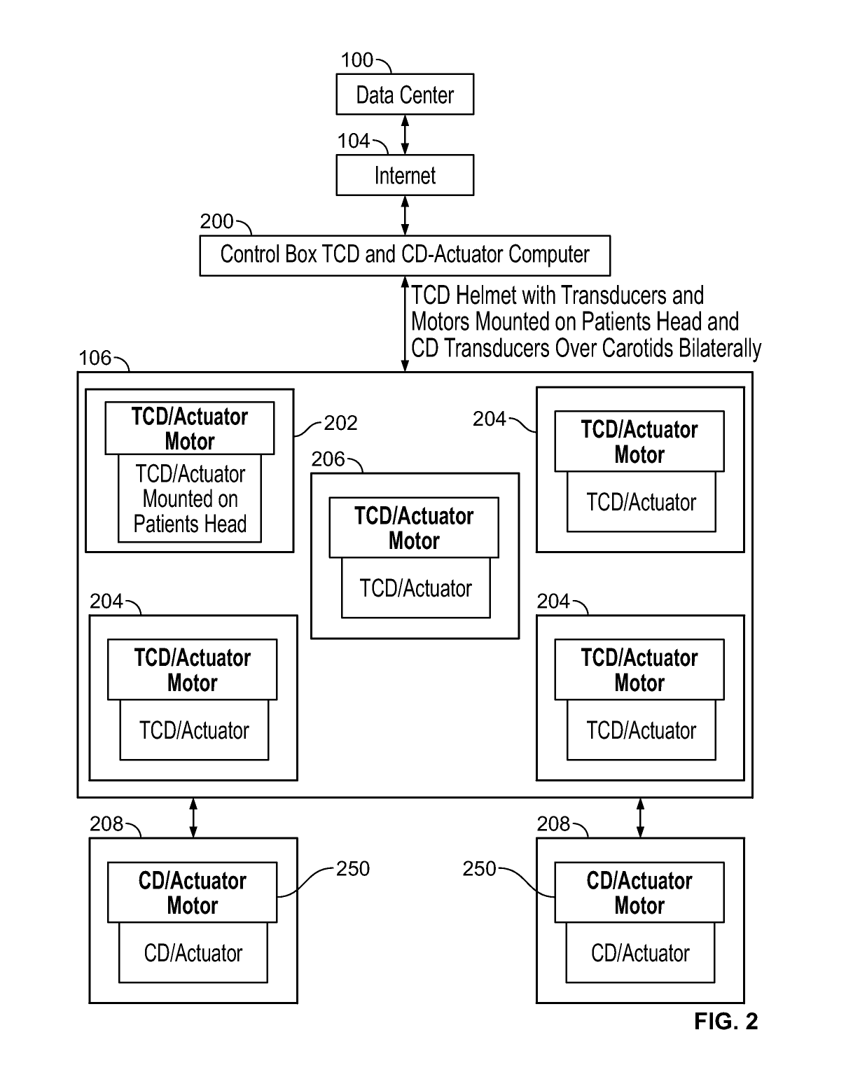

Referring now to FIG. 2, a simpler version of the system overview in FIG. 1, with a more detailed depiction of the helmet device 106 coupled with internet-controlled Transducers 300, is shown. The helmet device 106 contains transducers and actuators 300, controlled by micro-motors 250, and is mounted on the patient's 112 head. The transducers 300 are coupled to the micro-motors 250, allowing the position and angle of the transducers 300 to be controlled remotely over the internet, from the Data Center for example. The transducers and actuators 300 are driven by pulsed, oscillating currents from a TCD control box 200, which emits ultrasound waves. The TCD control box 200 can be turned on remotely. Operators (whether remotely or at the site) will move the TCD transducers 300 until they detect return Doppler signals from the TCD transducers 300.

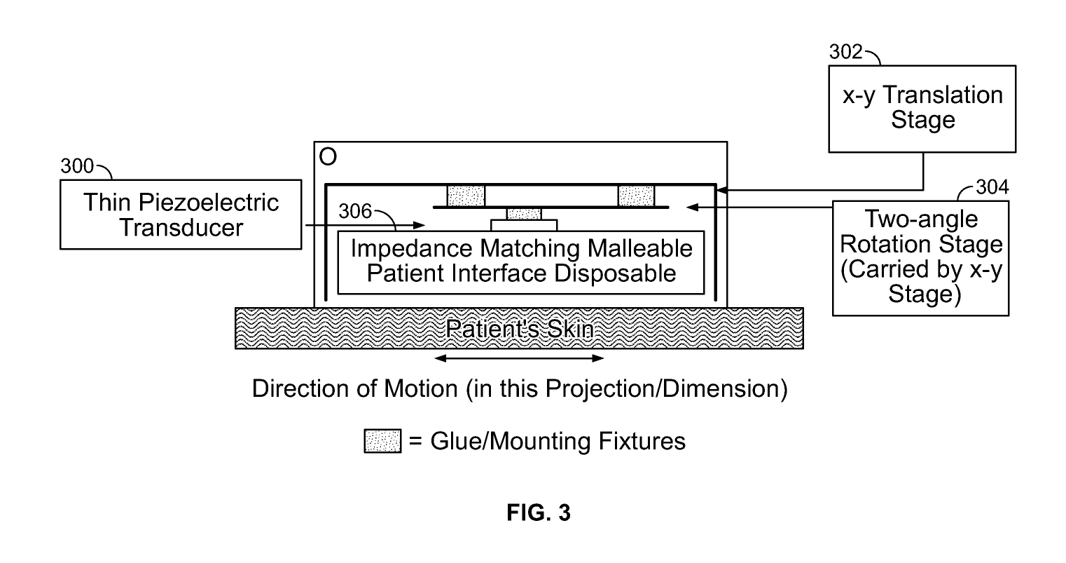

A map of the local thickness of the skull can be made by moving the TCD transducers 300 in x and y directions in a plane parallel to the local surface of the skull. The ultrasound waves travel into an impedance matching system (number) (shown later in FIG. 3), and then into the skull. On entering the skull, some ultrasound is reflected, and when the ultrasound exits the brain and enters the soft tissue of the skull, ultrasound is reflected again. The time between these two pulses can be converted into a distance. The speed of sound in the skull is known to be 3,360 meters/second, and multiplying the time interval between these pulses by the speed of sound gives the local thickness of the skull. Such thicknesses can be recorded and put into an array in computer memory, or a file on disc, or other media that then describes skull thicknesses. This helps establish an absolute reference frame for all work and in addition, alerts the operator as to where the thinnest parts of the skull are for easier insonification. A computer at the ambulance or emergency room or remote site 100 allows significant control of the TCD pulses, pre-processing of TCD data, aligning the TCD data to an absolute reference frame, and also relaying real-time images of the Doppler signal, synchronized with the pulse and the estimated depth and position of the insonated vasculature.

In another exemplary embodiment, the TCD control box 200 could contain a computer/Actuator computer or like means for viewing and sending images and information obtained from the transducers to the remote center.

Referring now to FIG. 3, a detail of the stage that enables movement of the transducer 300 on a soft disposable 306 is depicted. The movement of the transducer 300 on the soft disposable matches impedance of ultrasound, and allows the transducer 300 to travel on a plane parallel to the skull, under remote control from the Data Center 100. The transducer 300 can be pointed by a two-angle pointing system 304 that is carried by the x-y stage 302 The impedance matching insert 306 is made of low friction material, with friction comparable to Teflon, so an ultrasound transducer 300 can move along an impedance matching insert 306 without jamming. In addition, this impedance matching insert 306 enables the ultrasound transducer 300 to point at large angles to the normal vector of the skull, enabling the remote technician to thoroughly search for arteries.

In another exemplary embodiment, the impedance matching insert 306 can also be disposable.

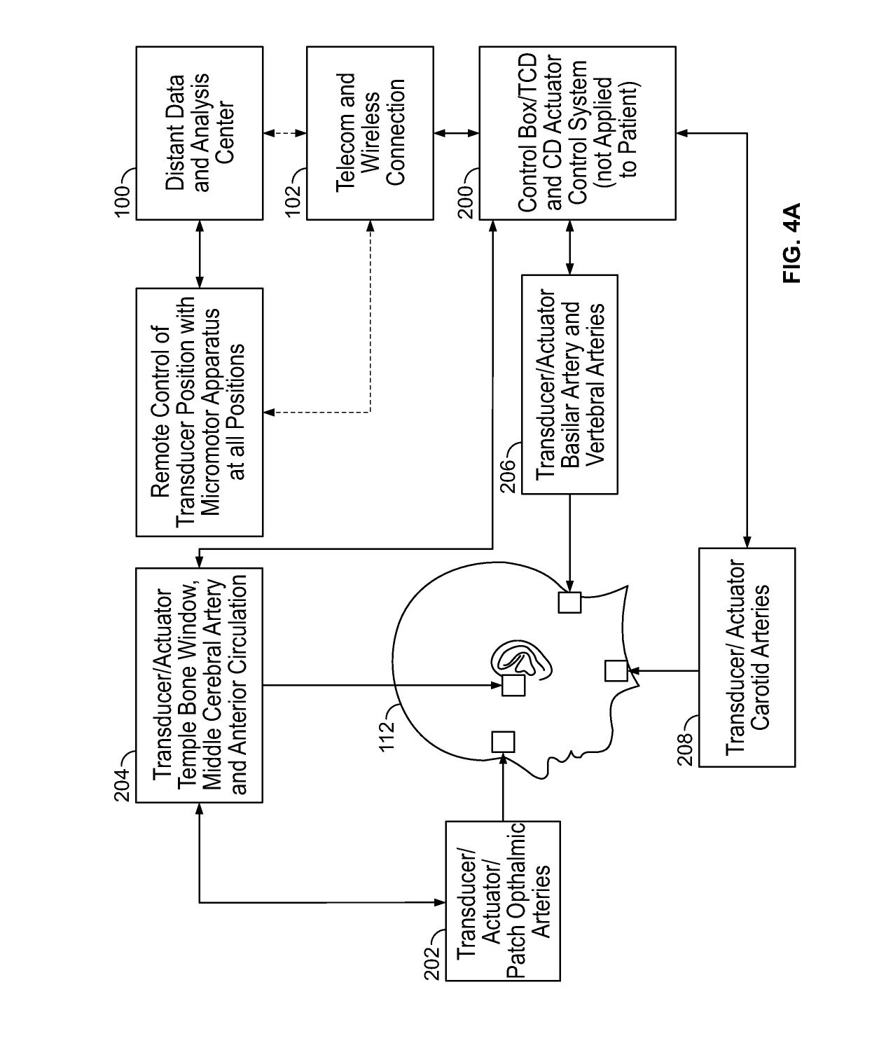

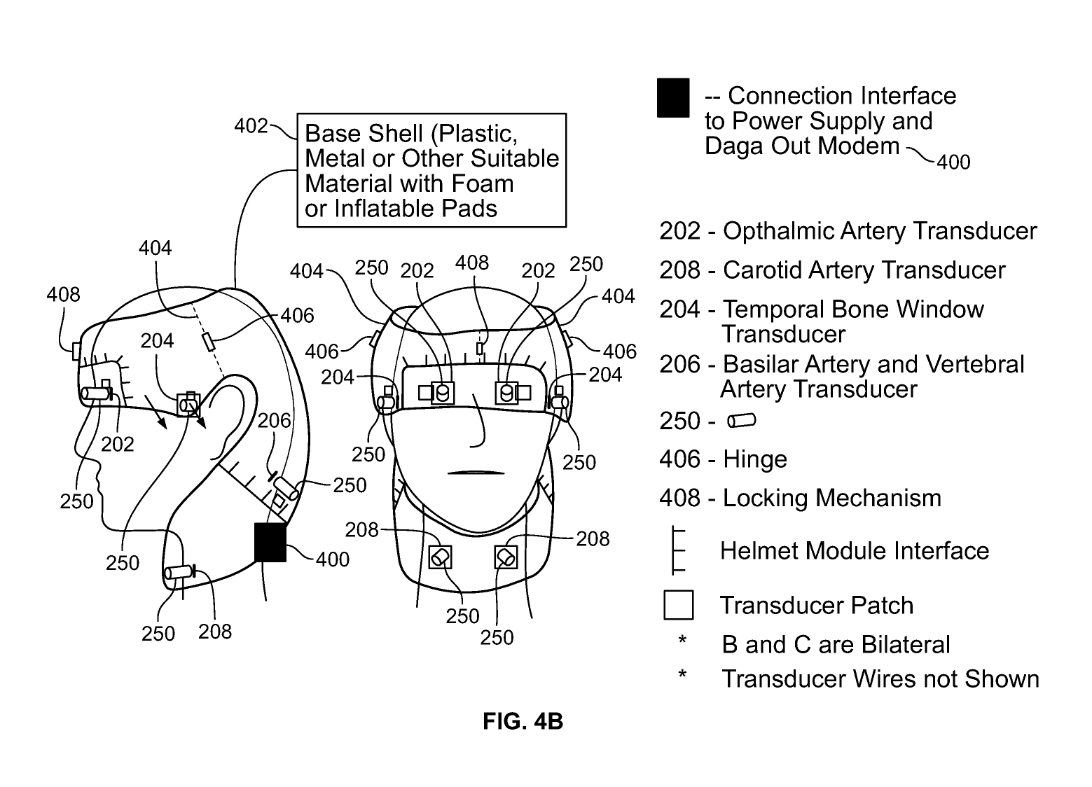

Referring now to FIG. 4a, an overview of the TCD/Actuator module placements on a patient's 112 head is depicted. The overview is depicted without the helmet device structure, so the placement of the ultrasound transducers 300 can be clearly seen. In this embodiment, four transducer-actuator boxes are depicted; however there are 7 in total—the transducer-actuator box pictured around left eye 202, left ear 204, and left neck/carotid area 208, each have a similar transducer-actuator box in similar locations on the other side/right side of the face.

Each transducer-Actuator box controls an actuator/motor 250 that can move the ultrasound transducer 300 in two dimensions and also point in two angles, all the while staying in contact with the flexible impedance matching insert 306. Signals sent to control the transducers 300/actuator motors 250 from the Remote Data Center 100, are transmitted wirelessly 102 to a radio or receiver in the ambulance (or helicopter, airplane, emergency room, or the like) in digital form, and then are routed to the TCD control box 200. The TCD control box 200 then transmits and receives signals to the transducers 300 and the actuators, enabling the remote operator to visualize the output of the TCD at a given position and angle. In an exemplary embodiment, each actuator/transducer 300 can be placed together inside of a transducer-actuator box.

Referring now to FIG. 4b, a 3-D view of the Trans-Cranial Doppler with remote control device is depicted. The device consists of a rigid base shell or helmet 402 that is either placed around the patient's 112 head or the head is placed into the base shell 402 while the base shell 402 is affixed to the supporting surface upon which the patient 112 is laying. The main material for the base shell 402 is a plastic, metal, or other like material that can be produced by thermoforming, injection molding, casting or other like means. The base shell 402 creates the framework for the helmet.

A conformable interior material for the base shell 402 allows a variety of head sizes and shapes to be accommodated for proper fit. This conformable material may be an open-cell or closed-cell foam, or an inflatable network of chambers, or a combination of both. Two pivoting shell members 404 (each hinged at 406) lock together to form the front of the helmet, and are held open during the insertion of the patient's 112 head, by a spring load or other like structure/device 406 These members 404 are then manually closed by a technician and locked together at the front 408 to form the helmet's fully encompassing shell.

At this time the basilar artery and vertebral artery transducer 206 and temporal bone window transducers 204 are in initial contact with the patient 112. Two separate modules are installed using a snap insertion interface that places the eye ultrasound transducers 202 and Carotid ultrasound transducers 208 over the eyes and over the carotid arteries. The ultrasound transducers over the carotid arteries 208 are applied using pressure sensitive adhesive in the appropriate locations, similar to EKG electrodes. These modules and members are of the same material construction as the base shell 402.

The helmet, in its final configuration, holds an array of seven custom Doppler transducers in lateral symmetry of the head, of which the two Carotid ultrasound transducers 208 and two temporal bone window ultrasound transducers 204 are bilateral, and of which the two eye ultrasound transducers 202, two temporal bone window ultrasound transducers 204, and basilar artery and vertebral artery ultrasound transducer 206 are remotely controlled in X-Y translation across the surface of the skull, pressured against the patient's 112 head in the Z direction at an appropriate pressure, pitch, and yaw angle rotation to aim the Doppler beam. These movements are carried out by a set of motor actuators 250, specified schematically in the drawing as TCD/Actuator Motor modules 250. The two eye transducers 202 and two carotid transducers 208 may be located on a more flexible substrate of the modules they are attached to, in order to prevent harmful levels of pressure applied to the eyes and the carotid arteries. In another exemplary embodiment, the two eye transducers 202 and two carotid transducers 208 may also be attached with adhesive. The wiring for the motor modules 250 and the transducers are routed through the helmet to the base of the shell, where the connection interface to the power supply and data output modem 400 is located. The separate helmet members have electronic connections in the snap interface to route power and data through the helmet's wiring.

The power supply and all data processing and transmission are done outside of the helmet to minimize the weight and complexity of the helmet. Each transducer can be used in series, activating only one at a time, or can be used in parallel to establish reference frames and use advanced signal processing, as described above. Alternatively, each set of transducers is operated jointly to be able compare and contrast signals. The operator is a skilled TCD technician or medical doctor in a command center that has a data link to the helmet attached to the patient 112 in the ambulance or other remote facility. The system will allow the remote operator to give real-time feedback on the patient's 112 cranial circulation status.

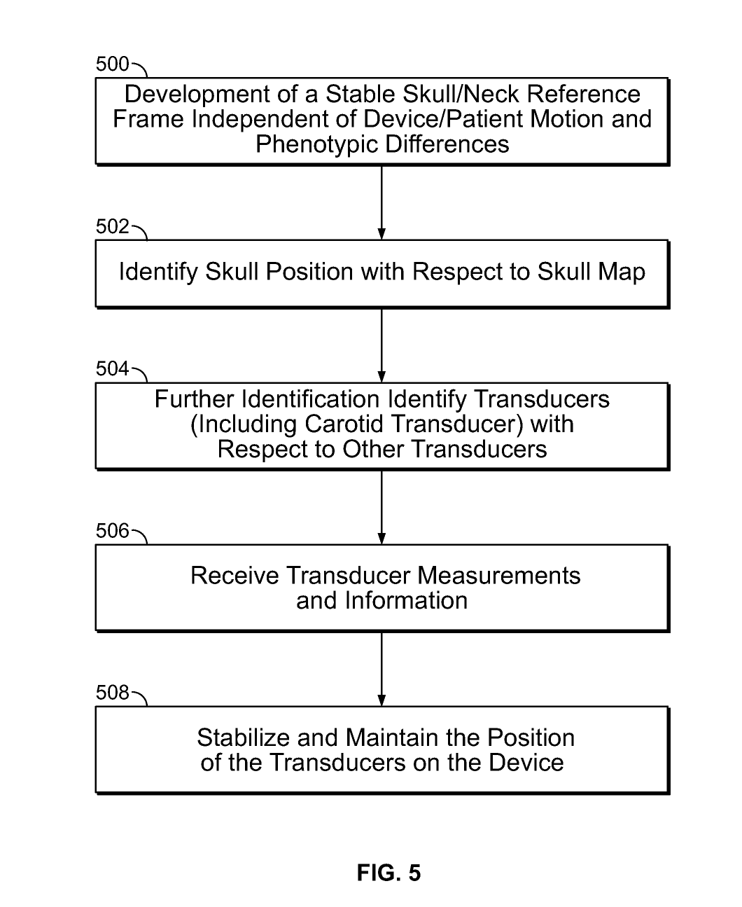

Referring now to FIG. 5, a hierarchal chart illustrating the development procedure of a stable skull/neck reference frame independent of the helmet device 106/patient's 112 motion and phenotypic differences is depicted. This chart illustrates the flow of data from the transducers 300 to the TCD control box/computer 200 system, so that a stable reference frame of the patient's 112 skull can be developed. After an initial identification of the position of the skull with respect to the skull map using both x-y scans 502, the control box 200 system further identifies the location of the transducers with respect to the other transducer 504. Next, the control 200 system receives the x-y scan information (transducer measurement information) 506, and uses the information to stabilize, i.e. measure and maintain its best position 508. The reference frame is set by the arrival times of ultrasound from the other transducers on the head; this enables freedom from mechanical disturbances, as for example in a bouncing ambulance, and also enable a reference frame that can be determined to sub-millimeter accuracy. The novelty of this “good positioning” overcomes traditional “transducer-movement noise,” which limits the size of vasculature or other structures to be larger than the rms of the ultrasound transducer movement time

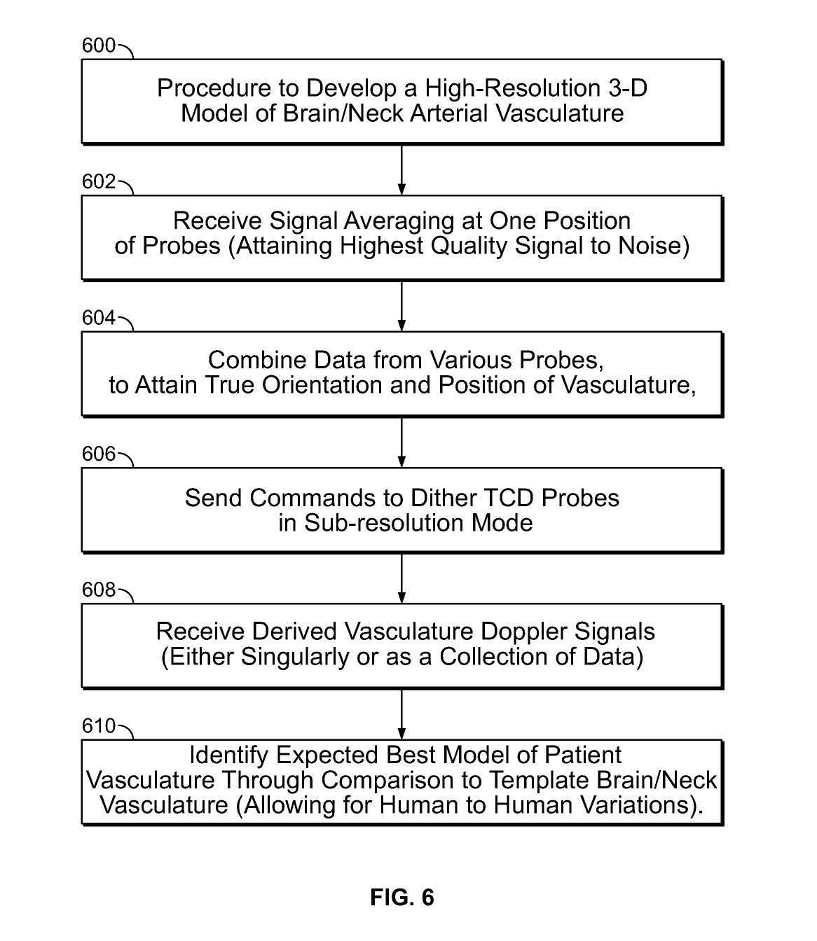

Referring now to FIG. 6, a decision tree showing the procedure used to develop a higher resolution 3-D model of a brain/neck arterial vasculature is depicted. The tree shows the algorithms for reduction of the signals to a high-quality set of images of brain arterial vasculature. These algorithms are coupled to the ultrasound transducer position as discovered by its position relative to skull vasculature, and also the arrival times of signal from other transducers that are depicted in FIG. 5. The TCD control box 200 system receives signals from one ultrasound transducer/position in the brain and averages it 602. Then TCD control box 200 system accumulates consecutive Doppler signals from each position in the brain, and co-adds them 604. If there is signal present, the signal should grow proportional to the number of insonation return signals captured, and the noise should only grow as the square root of the number of insonations captured.

In particular, if the ultrasound transducers are stepped at 1/10 of their resolution and high quality data is accumulated, then a mathematical fit (least squares or other minimization algorithm) can be made between the measured data and the underlying assumed vasculature which could give rise to such a number of dithered data images 608. In particular, for large vessels, skull vasculature is well known and easy to detect, and good starting point models can be estimated and used as an initial system to fit to 610. This algorithm is flexible enough to allow discovery of complete occlusions or hemorrhages, thus the fit also accommodates such medically important circumstances.

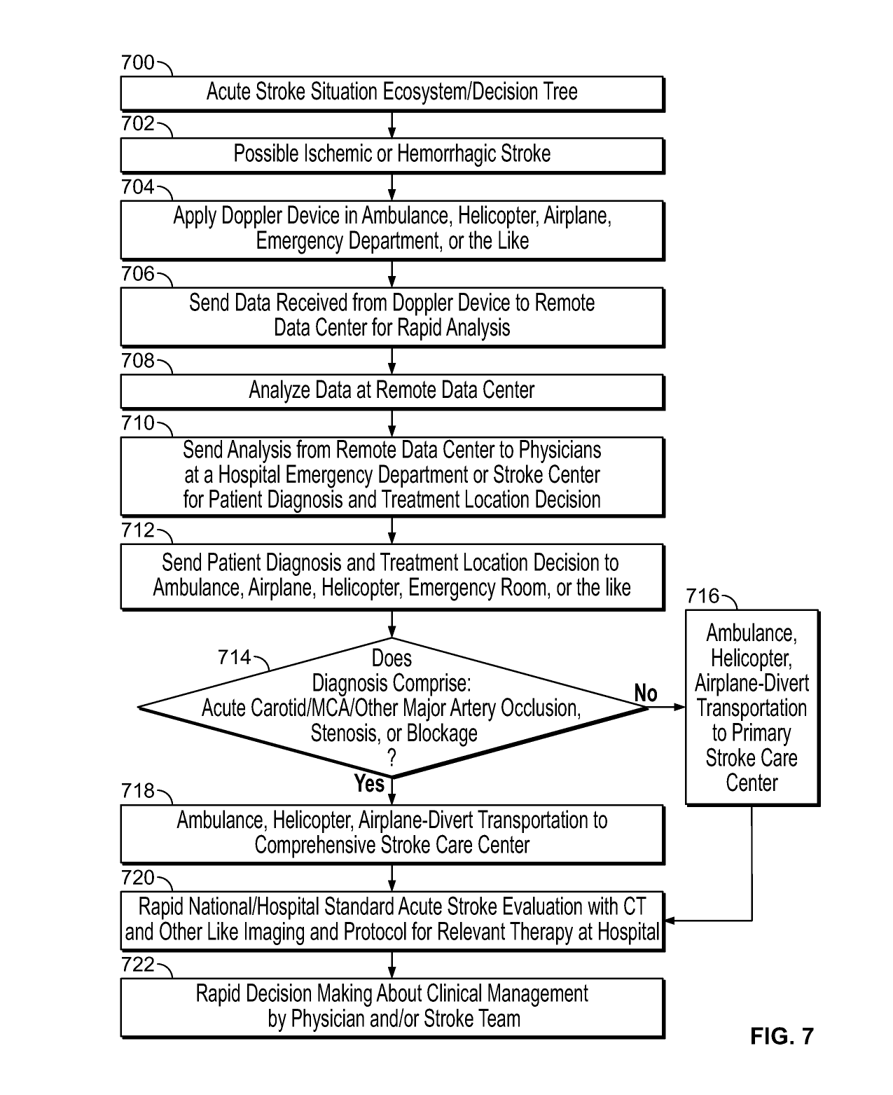

Referring now to FIG. 7, an acute stroke situation ecosystem/decision tree 700 is depicted. Once a patient 112 with a possible ischemic or hemorrhagic stroke is identified 702, the emergency providers at the patent pickup site use the helmet device 106 to speed along the analysis/diagnosis. In further exemplary embodiments, the patient site can be the residence of the patient 112, an ambulance, airplane, helicopter, emergency room/department, or the like. The emergency providers for example, would palpate the carotid arteries and apply the carotid Doppler transducers 208 as well as the helmet with the bitemporal window transducers 204 in front of both ears (accessing the middle cerebral arteries), the basilar artery and vertebral artery transducers 206 at the bottom back of the skull, as well as the ophthalmic arteries transducers 202 at the patient's eyes 704. In another exemplary embodiment, additional transducers accessing other arteries can be implemented. All transducers 300 are coupled to motors/actuators 250 which are coupled to the helmet device 106, and have the ability to be controlled remotely and wirelessly 102 from a remote data center 100. In order to maximize vascular signals, positioning of the transducers involves beam steering as well as remote transducer positioning.

Raw data from the transducers 300 is transmitted 706 wirelessly 102 from the patient 112 and device/invention 106 to the remote data center 100. The data is then processed 708 and rapidly analyzed by experts at the data center 100. Once analyzed, the information is rapidly transmitted 710 to physicians, stroke teams, or other like-experienced decision makers of the like, who are located where the patient 112 is being transmitted to, or at another hospital site, or the like. In another exemplary embodiment, the transmission of this data from the remote data center to physicians may be transmitted via internet, telephone, video teleconferencing or the like. The physicians can then use the data analysis sent from the remote data center 100 and other information sent regarding the patient 112, to aid them in deciding where the patient 112 should be transported to (i.e. stroke ready hospital, closest emergency department, primary stroke center, comprehensive stroke center, a transport plan from one location first to another, or the like.) 712.

Because the helmet device is evaluating large and medium size neck and brain blood vessels, it can give direct information on acute obstruction, occlusion, narrowing of these vessels, or the like. Such direct information can be essential in providing physicians with key information to appropriately and rapidly decide on the location to take the patient 112; with every second passed being a critical one. As just explained, the primary decision made by the physicians (or stroke team or other like-experienced decision makers), is whether the patient 112 has an acute carotid/MCA/or other major artery occlusion, stenosis, blockage, or the like 714. If the patient is decided to not have an acute carotid/MCA/or other major artery occlusion, stenosis, blockage, or the like, the physicians, for example, may decide that the patient should be transported to a primary stroke center for evaluation 716 for intravenous clot buster or other therapies, or to a stroke ready emergency department or other hospital where after appropriate evaluation by imaging and neurological examination can be performed. If the physicians decide that the patient 112 does have an acute carotid/MCA/or other major artery occlusion, stenosis, blockage, or the like, they may decide that the patient 112 should be transported to a comprehensive stroke center for evaluation 718.

Once transported to the location, rapid national/hospital standard acute stroke evaluation with CT and other like imaging and protocol will be conducted for relevant therapy at the hospital 720. Meanwhile, physicians, stroke team members, or other like experienced decision makers at the location the patient is transported to, will continue to analyze patient 112 and make rapid decisions regarding the patient, the treatment that should be done, whether transportation to another location should be done, etc. 722.

In an exemplary embodiment, the helmet device may also be used again, for the first time, or as many times as needed, at the new location the patient is transported to.

In an exemplary embodiment of this invention, the helmet device could aid in in clot lysis/removal decisions and safer and more thorough care of actual stroke.

In yet another exemplary embodiment is the possibility of linkage of the helmet device with stroke telemedicine. Physiological data from the device could be combined with a neurological evaluation, including but not limited to an NIH stroke scale. This would involve an appropriate and approved video and audio device as well and more strict criteria.

In yet another exemplary embodiment, video/audio stroke telemedicine input would be applied with the helmet device.

In yet another exemplary embodiment, the helmet device could be used in conjunction with CT scanners, CT/MR angiography, CT/MR perfusion, different weighted imaging MR scanning, telestroke devices, or other like evaluation therapies on ambulances, helicopter, airplanes, emergency rooms/departments, or the like.

In yet another exemplary embodiment, the helmet device could be used in combination with phased array with the transducers, with the ability to add additional transducers, for photoacoustic imaging that can provide data on diffusion perfusion mismatch. Brain tissue that is dead or irreversible would be differentiated from potentially retrievable tissue (penumbra).

In yet another exemplary embodiment, in non-acute stroke settings, this helmet device could be applied remotely and analyzed with or without telehealth services to evaluate and convey information to physician and medical providers nationally and internationally, including but not limited to doctor's offices, rural and urban health clinics, and rehabilitation and chronic care facilities.

While methods, devices, compositions, and the like, have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims. It is understood that the term, present disclosure, in the context of a description of a component, characteristic, or step, of one particular embodiment of the disclosure, does not imply or mean that all embodiments of the disclosure comprise that particular component, characteristic, or step.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps. Such terms should be interpreted in their most expansive forms so as to afford the applicant the broadest coverage legally permissible.

1. A device for detecting emboli in the brain comprising, in combination:
an array of ultrasound transducers;
actuators coupled to the array of ultrasound transducers;
said actuators enabled to alter, skew, move, rotate, or change the position of the transducers;
said actuators enabled to be controlled remotely; and
the array of ultrasound transducers having the ability to learn and send Doppler shifted signals, regarding blood flow from brain and neck vasculatures, to a remote site.

2. The device according to claim 1, further comprising:
means for wireless remote control capability and remote manipulation of the ultrasound transducers; and
means for wireless transmission and receipt of the Doppler signals over internet, radio, land links, and related systems.

3. The device according to claim 1, wherein the array of ultrasound transducers are mounted on x-y position stages and two angular pointing stages.

4. The device according to claim 3, wherein the ultrasound transducer's x-y and two angle positions can be controlled from a remote location via the internet, radio, land links, and related systems.

5. The device according to claim 1, wherein the actuators comprise robotic arms or other robotic manipulation systems that enable an ultrasound transducer to: move in space, approach and make contact with the patient's head, and begin searching for arterial Doppler signals.

6. The device according to claim 1, wherein the array of ultrasound transducers are single channel or phased.

7. The device according to claim 1, further comprising means for making a 3-dimensional model of the blood flow of the brain, using at least a Super-resolution algorithm and angular positions from an ultrasound transducer encoder and a signal return time from a vasculature.

8. The device according to claim 7, wherein remote control of the ultrasound transducers is done by way of feedback signals, from the device to the remote center and from the remote center to the device, in raw or analyzed form.

9. The device according to claim 1, further comprising ultrasound impedance matching inserts.

10. The device according to claim 9, wherein the ultrasound impedance matching inserts comprise:
materials made of intermediate sonic indices of refraction;
disposable materials (i.e. the insert as a whole itself is disposable); and
a smooth surface enabling the transducers to move, friction-free, over the surface of a patient's skull.

11. The device according to claim 1, further comprising a convex probe array used to image vessels from one position, for carotid artery insonation.

12. The device according to claim 1, wherein the ultrasound transducers comprise Transcranial Ultrasound Transducers.

13. The device according to claim 12, wherein the ultrasound transducers further comprise Carotid Doppler Ultrasound Transducers.

14. A device according to claim 13, wherein the Carotid Doppler Ultrasound Transducers comprise:
a B mode;
pulsed wave;
color flow monitoring;
power Doppler;
M mode;
automatic measurement;
triplex mode with B mode;
Pulsed Doppler; and
Color mode in real time.

15. A method for detecting emboli in the brain and sending the relevant data to a remote site which comprises, in combination:
configuring an ultrasound array to transmit a beam pattern sufficient to isonate a region of interest at an internal site of a subject;
finding, creating, and displaying maps or images of said region of interest;
identifying acute occlusion or stenosis in major brain and neck arteries;
wirelessly transmitting data identified to a remote site; and
wirelessly receiving information about data identified from the remote site at the site of the subject where the device is being used.

16. The method according to claim 15, wherein configuring an ultrasound array to transmit a beam pattern sufficient to insonate a region of interest at an internal site of a subject, said method comprises the steps of:
a) providing an array of ultrasound transducer elements;
b) outputting a beam pattern from said array of ultrasound transducer elements to insonate a region of interest at an internal site in a body that is sufficiently large that the beam output pattern comprises a multi-beam pattern,
insonating multiple receiver elements over a substantially simultaneous period by directing energy produced by said array of ultrasound transducer elements into said region of interest in said body, and
adjusting an amplitude of energy output by said array of transducers to cause the beam pattern output to have a generally flat upper pattern, nulling in a grating lobe region; and,
c) introducing a propagation time delay of the beam pattern output from said array of ultrasound transducer elements, wherein the propagation delay increases as a distance increases from a central output area of said array of ultrasound transducer elements.

17. The method according to claim 16, wherein the transmission of said array of transducer elements is configured in step b) comprising a beam pattern output by said array of transducer elements propagating from a point source having a focal distance located behind said array of transducer elements when viewed from said region of interest.

18. The method according to claim 16, wherein step b) further comprises adjusting a duty cycle of one or more pulses output by at least one transducer element of said array of transducer elements.

19. The method according to claim 16, wherein step b) comprises adjusting a quantity of pulses output by at least one transducer element of said array of transducer elements.

20. The method according to claim 17, wherein step b) further comprises adjusting a quantity of pulses output by said at least one transducer element of said array of transducer elements.

21. The method according to claim 16, wherein said array of transducer elements comprises an 8×8 array.

22. A method of configuring an ultrasound array to transmit a beam pattern sufficient to insonate a region of interest at an internal site of a subject, said method comprising, in combination:
a) providing an array of ultrasound transducer elements;
b) outputting a beam pattern from said array of ultrasound transducer elements to insonate a region of interest at an internal site in a body sufficiently large that the beam pattern comprises a multi-beam pattern,
insonating multiple receiver elements over a substantially simultaneous period by directing energy produced by said array of ultrasound transducer elements into said region of interest in said body, and
adjusting an amplitude of energy output by said array of transducers to cause the beam pattern output to have a generally flat upper pattern and nulling in a grating lobe region; and,
c) introducing a phase shift of the beam pattern output from said array of ultrasound transducer elements, wherein a degree of phase shift increases as a distance increases from a central output area of said array of ultrasound transducer elements.

23. The method according to claim 22, comprising:
moving the ultrasound transducer elements across the skull without extending the transducers in any direction abnormal or unparallel to the skull; and,
enabling the transducer to turn 90 degrees within its cable.

24. A method of configuring an ultrasound array to transmit a beam pattern sufficient to insonate a region of interest at an internal site of a subject, said method comprising the steps of:
a) providing an array of ultrasound transducer elements;
b) outputting a beam pattern from said array of ultrasound transducer elements to insonate a region of interest at an internal site in a body sufficiently large that the beam pattern comprises a multi-beam pattern,
insonating multiple receiver elements over a substantially simultaneous period by directing energy produced by said array of ultrasound transducer elements into said region of interest in said body, and
adjusting an amplitude of energy output by said array of transducers to cause the beam pattern output to have a generally flat upper pattern and nulls in a grating lobe region; and,
c) introducing a phase shift of the beam pattern output from said array of ultrasound transducer elements, wherein a degree of phase shift increases as a distance increases from a central output area of said array of ultrasound transducer elements.

25. The method according to claim 24, wherein the transmission of said array of transducer elements is configured in step b) such that the beam pattern output by said array of transducer elements appears to propagate from a point source having a focal distance located behind said array of transducer elements when viewed from said region of interest.

26. The method according to claim 24, wherein step b) further includes adjusting a duty cycle of one or more pulses output by at least one transducer element of said array of transducer elements.

27. The method according to claim 24, wherein step b) includes adjusting a quantity of pulses output by at least one transducer element of said array of transducer elements.

28. The method according to claim 24, wherein said array of transducer elements comprises an 8×8 array.

29. A method for operating an array of ultrasound transducer elements, wherein:
the element spacing in the array is greater than, equal to or less than a half wavelength of the ultrasound energy produced by the elements, and
wherein the array is used differently in transmit and receive modes, comprising:
forming a transmit beam from a position external to a region of interest encompassing a plurality of receive beams and initially acquiring a signal by insonating a target region comprising multiple receive beam positions over a substantially simultaneous period;
receiving data from the multiple receive beam positions of the array;
combining the received data in a processor;
locking onto the receive beam and the point(s) producing a peak signal; and
correcting for motions in the target region by periodically forming multiple receive beams and re-acquiring the peak signal.

30. The method of claim 29, additionally comprising forming a transmit beam using a sub-array of the array.

31. The method of claim 29, wherein the large target region is a 3-D spatial region.

32. The method of claim 29, wherein the transmit beam uniformly insonates over a 2-D transmitter sub-aperture.

33. The method of claim 29, wherein the transmit beam has a fixed focus.

34. The method of claim 29, additionally comprising simultaneously and digitally forming multiple receive beams for receiving data.

35. The method of claim 29, additionally comprising Doppler processing the received data.

36. The method of claim 29, wherein the array is a monostatic array, and additionally comprising transmitting from the full aperture and scanning the transmitted beam over the region being examined.

37. The method of claim 29, further comprising using a transmitter diversity technique to spread temporal intensity over the face of the array.

38. The method of claim 37, comprising using a different transmit sub-aperture for different coherent burst of pulses.

39. The method of claim 29, further comprising steering the receive beams to a point or points that produce a peak signal.

40. The method of claim 39, wherein the peak signal is a maximum amplitude at high Doppler frequencies.

41. The method of claim 29, further comprising steering the receive beams using a phase steering or time-delay steering technique.

42. The method of claim 29, further comprising providing the array of ultrasound transducer elements on a low-profile easily-attached transducer patch.

43. The method of claim 29, further comprising determining spatial coordinates of received data,
measuring a velocity of the blood flow in each frequency;
collecting a data point of said velocity;
measuring a velocity of the blood flow at the next frequency; and
making a plot of the velocity in the resolution element.

44. The method of claim 43, further comprising forming and displaying a 3D map based on the spatial coordinates of received data.

45. The method of claim 43, wherein the received data comprises time delay to the reflected signal, the velocity of the structures, and the angular positions of the structures.

46. The method of claim 43 wherein the data could be collected and received from one transducer or multiple transducers.

47. The method of claim 46 wherein each transducer comprises:
a characteristic angle;
a characteristic Doppler shift; and
a characteristic depth for each artery detected.

48. The method of claim 46 wherein:
the data from multiple transducers comprise depth data and Doppler shift data; and,
the data are combined to form a best fit model of the brain vasculature.

49. The method of claim 48, wherein the data can be fit to a template image of a typical brain vasculature via at least squared minimization way, or maximum likelihood, or other like procedure.

50. The method of claim 49, wherein the template image would take into consideration, varying sizes of patient's skulls and vascular positions via a method comprising:
finding major brain and neck arteries first; and,
using the major brain and neck arteries findings to discover and find smaller vasculatures.

51. The method of claim 29, further comprising tracking of arteries capabilities comprising the method of:
scanning the ultrasound transducer in various directions and angles; and,
following the angle of maximum signal.

52. The method of claim 29, further comprising a scan mode of super resolution comprising:
stepping the transducers at a fraction of resolution element (ex: 1/10 of resolution element);
fitting the resulting signal to a “super-resolution image” of the acquired signals (ex: of the acquired 10 signals); and
making a measurement of the width of the velocity distribution in one normal resolution element/voxel.

53. The method of claim 52, wherein the measuring of the width of the velocity distribution in one normal resolution element/voxel, comprises the method of:
measuring a velocity of the blood flow in each frequency;
collecting a data point of said velocity;
measuring a velocity of the blood flow at the next frequency; and
making a plot of the velocity in the resolution element.

54. The method of claim 53 wherein velocity fields are coupled with pulse modulation data to determine characteristics of vasculatures in various regions of the brain.

55. The method of claim 29, further comprising forming and displaying a map of the skull thickness at a given x-y position on the skull.

56. The method of claim 51, wherein computing the skull thickness comprises finding a time delay and converting it to compute the thickness of the skull at that point.

57. The method of claim 51, further comprising alternately using a tone or other audible or visual means such as acoustic impedance or electronic detection of specific chirp) to find skull thickness via remote operator (i.e. operators at a remote site).

58. The method of claim 52, wherein finding a time delay comprises:
measuring a first pulse from an initial reflection of a pulse from the ultrasound transducer; and
measuring a first pulse from the second reflection of the pulse when the transducer pulse exits the skull bone and enters the brain.

59. The method of claim 29, further comprising finding and using the path through the skull with the least amount of bone material when a large angle is needed to reach a vasculature.

60. The method of claim 29, further comprising a method for setting up and measuring an absolute reference frame upon the head:
understanding the exact position on the head and relative to the head using signals from multiple transducers and reflection signals;
discovering our position on the skull;
measuring time delays between the other transducers; and
constantly finding and monitoring the exact placement of the head frame at all times.

61. The method of claim 29, further comprising time-tagging every signal received or sent from the transducers, thus enabling an absolute and stable reference frame to less than at least about one millimeter accuracy.

62. The method of claim 29 further comprising:
at least a Signal Averaging Mode which comprises the method of:
determining the angular positions by determining the angles of the ultrasound transducers and the super-resolution position; and
accumulating data for every resolution element in the brain.

63. The method of claim 29 further comprising insonating positions of interests for longer periods until a signal is built up against a background.

64. The method of claim 29 further comprising enhancing signal to noise by successively scanning over regions of interest with super resolution.

65. The method of claim 29 wherein the signal to noise increases approximately proportional to the square-root number of scans or the square-root of time.

66. A data reduction and analysis system wherein actuators coupled to ultrasound transducers can be remotely manipulated, over the Internet or radio or land links, with control taking place at a remote site distal from the patient.

67. The system according to claim 66 wherein said actuators may comprise robotic arms or other robotic manipulation systems that enable a TCD probe to move in space, approach and make gentle contact with the patient's head, and begin searching for arterial doppler signals.

68. The system according to claim 66 further comprising means to:
make maps of brain vasculatures;
identify acute occlusion or stenosis in major brain and neck arteries;
remotely send the data identified, to a remote site; and
provide capabilities to quickly analyze the data identified and advise diversion of the patient to a primary or comprehensive stroke center upon finding of a collusion in major arteries or blockages of the carotid or major cerebral or vertebral arteries.

69. A method to detect prostate cancer, comprising, in combination:
injecting ICN-pSMA molecule to the prostate area which combines with the surface of Prostate Cancer cells, and liberates ICN in the process;
insonating the prostate and the adjacent vicinity with energy from a phased array Doppler device;
insonating the prostate and the adjacent vicinity with energy from a photoacoustic device; and
detecting the spectrum of a plurality of ICN molecules in the region of the Cancer.
